(19) United States
(12) Patent Application Publication
(10) Pub. No.: US 2007/0265466 A1
(43) Pub. Date: Nov. 15, 2007

(54) Process for the coupled production of chlorline and isocyanates

(75) Inventors: Markus Dugal, Kempen (DE); Bernd Pennemann, Bergisch Gladbach (DE); Friedhelm Kamper, Krefeld (DE)

Correspondence Address: BAYER MATERIAL SCIENCE LLC, 100 BAYER ROAD, PITTSBURGH, PA, 15205, US

(73) Assignee: Bayer MaterialScience AG

(21) Appl. No.: 11/801,719

(22) Filed: May 10, 2007

(30) Foreign Application Priority Data
May 13, 2006 (DE) 102006022447.7

(51) Int. Cl. C07C 263/10

(52) U.S. Cl. 560/347

(57) ABSTRACT

A process for the coupled production of chlorine and isocyanates in which the sulfuric acid used for both processes is combined after use, concentrated together and returned to one or both processes.

### BACKGROUND OF THE INVENTION

The present invention relates to a process for the coupled production of chlorine and isocyanates in which the sulfuric acid used to produce each of these materials is combined after use, concentrated together and returned to the process for producing one or both of these materials.

Chlorine and nitrated aromatic compounds are important industrial intermediates that are needed, for example, for the production of polyurethane raw materials such as toluene diisocyanate (TDI) or methylene diphenyl diisocyanate (MDI). Chlorine gas is needed in this regard for the production of phosgene, which is used to produce isocyanates from amines. The hydrogen chloride (HCl) formed as a by-product during the phosgenation of the amine can be converted back to chlorine after various recycling processes. However, chlorine can also be produced from other raw materials such as sodium chloride by electrolysis methods.

Examples of the diverse industrial methods used for the production of chlorine include:
1. Production of chlorine in NaCl electrolysis processes.
2. Conversion of HCl to chlorine by electrolysis of aqueous HCl with diaphragms or membranes as the separating medium between the anode and cathode compartment. The by-product of this conversion is hydrogen.
3. Conversion of HCl to chlorine by electrolysis of aqueous HCl in the presence of oxygen in electrolytic cells with an oxygen depletion cathode (ODC). The by-product of this conversion is water.
4. Conversion of HCl gas to chlorine by gas-phase oxidation of HCl with oxygen at elevated temperatures on a catalyst. The by-product of this conversion is also water. This process known as the “Deacon process” has been in use for more than a century.

In most processes for chlorine production from both aqueous and gaseous HCl, after the reaction and optionally first processing steps (e.g., the absorption of unreacted HCl), the process gas undergoes a drying step to remove any water content which can interfere with subsequent processing steps (such as chlorine liquefaction or distillation) or applications. Concentrated sulfuric acid in the range from 90.0 to 98.0 percent by mass of sulfuric acid based on the total percent by mass of sulfuric acid plus water is used as the drying agent. Large amounts of dilute sulfuric acid with a concentration of from 70.0 to 89.9 percent by mass of sulfuric acid based on the total percent by mass of sulfuric acid plus water are formed. This dilute sulfuric acid must either be concentrated in a distillation plant with expenditure of energy or disposed of.

JP 2004-269408 and JP 2001-192647 describe methods for drying chlorine gas with sulfuric acid in drying columns in which sulfuric acid losses are reduced and split streams of used sulfuric acid are recycled by mixing with highly concentrated fresh sulfuric acid, but a concentration by distillation of the used sulfuric acid is not described. Furthermore, neither of these documents mentions that the concentrated or dilute sulfuric acid from the process can also form part of the nitrating acid in a nitration reaction.

U.S. Pat. No. 3,201,201 describes the drying of chlorine gas from HCl oxidation and the regeneration of sulfuric acid by adiabatic flash evaporation. Here too a further use of the sulfuric acid in a nitration reaction with nitrating acid is not described.

Mono- or dinitrated aromatic compounds such as nitrobenzene, dinitrobenzene, nitrochlorobenzene, nitrochlorotoluene, nitrotoluene and dinitrotoluene are, with a few exceptions, generally produced by means of nitration processes in which the aromatic starting compound (e.g., benzene, chlorobenzene or toluene) is reacted in a sulfuric acid-catalyzed two-phase reaction with nitric acid to form the desired mono- or dinitrated aromatic compound. A wide variety of process variants can be used, for example, isothermal or adiabatic nitration. A survey of the various common process and reactor types can be found in EP-A 0708076, for example.

In order to be reused, the sulfuric acid must be separated from the nitrated product after the reaction and concentrated by distillation, for example, with expenditure of energy. The sulfuric acid recovered after the reaction can be concentrated, depending on the desired target concentration, by means of a multi-stage vacuum distillation such as that described in U.S. Pat. No. 6,156,288 and DE-A 19 642 328, or by flash evaporation as described in U.S. Pat. No. 3,201,201.

The common methods for chlorine drying described in the prior art therefore result in large amounts of dilute sulfuric acid which must either be concentrated in a distillation plant with high expenditure of energy or disposed of via the waste-water from chlorine production. In the nitration of aromatics, the sulfuric acid used as catalyst is likewise concentrated after the reaction with high expenditure of energy in order to be reused. In this process, large amounts of sulfuric acid are also lost via the product/waste-water route, and this has to be replaced by fresh sulfuric acid. The sulfuric acid contained in the waste-water from the chlorine production and nitration process has to be neutralized using large amounts of sodium hydroxide solution before the waste-water can be sent to a sewage treatment plant.

Although U.S. Pat. No. 5,888,920 describes how, after an appropriate concentration, the dilute sulfuric acid produced in alkylation, chlorine drying or nitration can be returned to the reaction process from which it was obtained. U.S. Pat. No. 5,888,920 does not describe how a dilute sulfuric acid obtained from a nitration or chlorine drying process can, after combination of these two sulfuric acids and concentration, be returned to one or both without the need to consider from which process the original dilute sulfuric acid was obtained.

### SUMMARY OF THE INVENTION

The object of the present invention is to provide a process for the coupled production of chlorine and isocyanates that allows the dilute sulfuric acid obtained in both processes to be processed in a combined process without high energy costs and investment costs and with economy of neutralizing agents in such a way that it can be returned both to chlorine production and to the nitration reactions, regardless of the process from which the dilute sulfuric acid was originally obtained.

This object is achieved by a process for the coupled production of isocyanate(s) and chlorine in which the sulfuric acid used for both processes is combined after use, concentrated together and returned to one or both processes.

### DETAILED DESCRIPTION OF THE INVENTION

The present invention relates to a process for the coupled production of isocyanate(s) and chlorine comprising the following steps:
a) nitrating aromatic compounds such as toluene, halogenated toluene, benzene and halogenated benzene to form the corresponding mono- or dinitrated aromatic compounds using nitric acid in the presence of sulfuric acid at a concentration of from 65.0 to 98.0% by mass until the concentration of the sulfuric acid is reduced by from 0.5 to 25% by mass;
b) transferring the dilute sulfuric acid obtained from step a) to a sulfuric acid concentration plant;
c1) reacting the mono- or di-nitrated aromatic compound obtained from step a) with hydrogen to form the corresponding amine or diamine;
c2) if the amine formed in c1) is aniline, further reacting the aniline with formaldehyde to form the diamines and polyamines of the diphenylmethane series;
d) reacting the amine(s) produced in step c1) or c2) with phosgene to form the corresponding isocyanates in two mutually independent reactions;
e) transferring the aqueous dissolved or gaseous hydrogen chloride formed in the reaction of the amine(s) with phosgene to a chlorine gas production unit;
f) producing chlorine gas in the chlorine gas production unit;
g) removing the water of reaction or moisture from other sources from the chlorine gas (“drying”) obtained from step f) by treating the chlorine gas with a sulfuric acid having a concentration of 90.0 to 99.0 percent by mass of sulfuric acid, based on the total mass of sulfuric acid plus water, until the sulfuric acid has a concentration of 65.0 to 90.0 percent by mass of sulfuric acid based on the total mass of sulfuric acid plus water;
h) reacting the dry chlorine gas obtained from step g) with carbon monoxide to produce phosgene;
i) feeding the phosgene obtained from step h) to the phosgenation reaction conducted in step d),
j) transferring the dilute sulfuric acid obtained from step g) either to (i) the sulfuric acid concentration plant of step b) or (ii) to one or more nitration reactions being conducted in accordance with step a) with subsequent transfer of the further diluted sulfuric acids obtained from this/these nitration(s) to the same sulfuric acid concentration plant according to step b);
k) increasing the concentration of the combined dilute sulfuric acid streams from steps b) and g) in the sulfuric acid concentration plant by means of one or more vacuum distillation stages or to several different elevated concentrations by removing split streams after different distillation stages, as required for one or more of the process stages of aromatic nitration according to step a) or chlorine gas drying according to step g); and
l) returning the combined or separate split streams of concentrated sulfuric acid obtained from step k) for either full or partial use in step g) and/or step a).

The nitration of each individual aromatic compound in step a) is conducted in mutually independent reactors which are not connected to one another. The nitration is conducted in the presence of sulfuric acid. The concentration of the sulfuric acid is dependent upon the compound to be nitrated and the nitration level. Concentrations of from 65.0 to 98.0 percent by mass of sulfuric acid, based on the total mass of sulfuric acid plus water, are generally used to catalyze the nitration reaction. Upon completion of the nitration, the sulfuric acid is diluted and has a concentration that, depending on the amount of sulfuric acid used and the number of nitration stages, is 0.5 to 25 percentage points less concentrated than the initial concentration of the sulfuric acid used.

The process of the present invention is advantageous if the chlorine gas is produced in step f) by any of the following processes: NaCl electrolysis; HCl electrolysis by the membrane or diaphragm method; HCl electrolysis in electrolytic cells with an oxygen depletion cathode; and/or catalytic HCl oxidation with oxygen.

The process of the present invention is particularly advantageous when the nitration product(s) produced in step a) are nitrobenzene, dinitrobenzene, nitrochlorobenzene, nitrochlorotoluene, nitrotoluene and/or dinitrotoluene.

The process of the present invention is advantageous if the dilute sulfuric acid obtained by step g) is freed from residual chlorine and/or HCl down to a residual concentration of <1000 ppm Cl before being combined with other sulfuric acid in the sulfuric acid concentration plant.

The process of the present invention is particularly advantageous if the dilute sulfuric acid obtained by step g) is freed from residual chlorine and/or HCl down to a residual concentration of <10 ppm Cl before combination with other sulfuric acid in the sulfuric acid concentration plant.

The process of the present invention is advantageous if, before being returned to step 1) of the process according to the invention, either the dilute sulfuric acid obtainable from step a) or the concentrated sulfuric acid obtained by step k) is freed from impurities using: inorganic nitrogen compounds such as nitrosyl sulfuric acid down to a residual concentration of <0.3 wt. %; or using highly volatile organic compounds such as dinitrotoluene, nitrobenzene, dinitrobenzene, nitrophenols, nitrocresols, nitrobenzyl alcohols, nitrobenzaldehydes down to a residual concentration of <50 ppm; or using low-volatility organic compounds such as hydroxynitrobenzoic acid, nitrobenzoic acids or aliphatic carboxylic acids down to a residual concentration of <500 ppm; or using volatile sulfur compounds such as SO₂ down to a residual concentration of <30 ppm.

The process of the present invention is advantageous if toluene is used in step a) and after dinitration in step a) to produce dinitrotoluene using sulfuric acid with a concentration of 86.0 to 96.0 percent by mass of sulfuric acid (based on the total mass of sulfuric acid plus water) in the first nitration stage, which in the course of the second nitration stage is diluted to a concentration of from 80.0 to 85.9 percent by mass of sulfuric acid (based on the total mass of sulfuric acid plus water), the dinitrotoluene is then reacted in accordance with step c1) with hydrogen to form toluene diamine (TDA) which is then reacted with phosgene according to step d) to form toluene diisocyanate (TDI).

The sulfuric acid obtained from the second nitration stage having a concentration of from 80.0 to 85.9 percent by mass of sulfuric acid (based on the total mass of sulfuric acid plus water) is preferably returned once more to the first nitration stage of the process according to step a) and then diluted to a concentration of from 70.0 to 79.9 percent by mass of sulfuric acid (based on the total mass of sulfuric acid plus water) and this sulfuric acid is then fed to the sulfuric acid concentration plant in accordance with step b).

The process of the present invention is also advantageous if benzene is nitrated in step a) and after mononitration in step a) to obtain nitrobenzene using sulfuric acid with a concentration of from 69.5 to 72.5 percent by mass of sulfuric acid (based on the total mass of sulfuric acid plus water) which in the course of nitration is diluted to a concentration of 66.5 to 69.4 percent by mass of sulfuric acid (based on the total mass of sulfuric acid plus water), the nitrobenzene is then reacted with hydrogen to form aniline, which in turn is reacted with formaldehyde in the presence of an acid catalyst to form diamines and polyamines of the diphenylmethane series, which in turn are then reacted with phosgene to form the corresponding di- and polyisocyanates of the diphenylmethane series.

For the chlorine production element of the process according to the invention, any of the chlorine production processes known to the person skilled in the art can be used. Examples of known processes for the production of chlorine include: NaCl electrolysis; conversion of HCl to chlorine by electrolysis of aqueous HCl with diaphragms or membranes as the separating medium between the anode and cathode compartment; conversion of HCl to chlorine by electrolysis of aqueous HCl in the presence of oxygen in electrolytic cells with an oxygen depletion cathode (ODC); and conversion of HCl gas to chlorine by gas-phase oxidation of HCl with oxygen at elevated temperatures on a catalyst.

Particularly preferred is the production of chlorine by reaction of HCl gas by gas-phase oxidation with oxygen at temperatures in the range from 180° C. to 500° C. in a Deacon process or in a process derived from the Deacon process (as described, for example, in EP-743277-B1; DE 19734412-A1; DE 19748299-A1; DE 10242400; and DE 10244996) on a fixed catalyst containing at least one metal in elemental or bound form selected from Cu, Ru, Au, Rh, Pd, Pt, Os, Ir, Ag, Re, Ce, Bi, Ni, Co, Ga, Fe and Nd. The metals or metal compounds can be unsupported or be supported on, e.g., metal or semi-metal oxides such as aluminum oxides, silicon oxides, titanium oxides, zirconium oxides or tin oxides, on mixed metal oxides, on ceramic materials, on activated carbons, carbon blacks or carbon nanotubes, on metal or semi-metal carbides, on metal or semi-metal nitrides or on metal or semi-metal sulfides.

If catalytic gas-phase oxidation is used to react the hydrogen chloride formed in step e), the gaseous hydrogen chloride from the phosgenation process is generally transferred to the HCl oxidation process. Before gas-phase oxidation is performed with the hydrogen chloride gas, purification operations can optionally be carried out, for example to remove phosgene, residual solvent such as chlorobenzene or o-dichlorobenzene, chlorinated hydrocarbons, organic and inorganic sulfur or nitrogen compounds or carbon oxides such as CO or CO₂. Possible separation or purification operations that can be used are, for example, adsorption, adsorption/desorption, freeze condensation, compression, pressure distillation or selective oxidation. The catalytic gas-phase oxidation and processing of the product gas stream can be performed in accordance with any of the techniques known to those skilled in the art. Examples of such techniques are described in EP-B1 743277; U.S. Pat. No. 6,852,667; DE-A 19734412; WO 2005014470; DE-A 10244996; US-A 200411411; and EP-B 767138.

The nitration process of step a) can be used to produce a wide array of nitro aromatics, particularly those selected from the group of nitrobenzene, dinitrobenzene, nitrochlorobenzene, nitrochlorotoluene, nitrotoluene and dinitrotoluene. All processes for sulfuric acid-catalyzed nitration known to the person skilled in the art, such as that described for example in EP-A 0708076, can be used.

The coupling of one or more different chlorine production processes to one or more nitro aromatic production processes or isocyanate production processes is also possible.

The acid can be concentrated by any of the common processes of the prior art for removing water from sulfuric acid. Examples of these include single-stage or multi-stage vacuum distillation or flash evaporation, heating with microwaves or electromembrane methods. The use of single- or multi-stage vacuum distillation and/or flash evaporation is particularly preferred, however.

In a particularly preferred embodiment of the process of the present invention, concentrated sulfuric acid in the concentration range from 90.0 to 98.0 percent by mass of sulfuric acid (based on the total mass of sulfuric acid plus water) is used to dry the chlorine gas and the dilute sulfuric acid having a concentration in the range from 70.0 to 90.0 percent by mass of sulfuric acid (based on the total mass of sulfuric acid plus water) generated from the chlorine gas drying is fed to a sulfuric acid concentration unit in a nitration plant (e.g., a plant for the dinitration of toluene). The amount of acid supplied to the nitration/acid concentration process corresponds to all or part of the sulfuric acid losses from the nitration/acid concentration process.

In an alternative mode of operation, the sulfuric acid from the chlorine drying process can also be introduced directly into one or more reaction stages of the nitration process and used as a catalyst for the nitration reaction, the dilute sulfuric acid that is formed then being supplied to an acid concentration stage. In this way, by combining the processes, disposal and associated neutralization with sodium hydroxide solution or special processing of the sulfuric acid from the chlorine gas drying process is avoided and the amount of fresh sulfuric acid purchased for the nitration process is minimized.

Impurities in the dilute sulfuric acid from the chlorine production process according to step g), such as residual chlorine, can optionally be separated off before introduction of the dilute sulfuric acid into the sulfuric acid concentration plant in a nitration plant or a nitration process, in order to prevent corrosion and chlorine contamination in the nitration process. This purification of the sulfuric acid can take place by a wide variety of methods, such as those described in DE-A 2 063 592 (adsorption on activated carbon) or Khorasani et al., Journal of Bangladesh Academy of Sciences, 1982, 6 (1-2), 205-207 (stripping with air). To avoid corrosion problems, resistant materials such as the special nickel alloys disclosed in JP 2006045610 can also be used in the sulfuric acid concentration plant. Removal of residual chlorine by stripping is preferred. Here the sulfuric acid is freed from residual chlorine and HCl so that a residual chlorine concentration in the range of <1000 ppm, preferably less than 100 ppm, most preferably less than 10 ppm, is obtained.

In another embodiment of the present invention, the dilute sulfuric acid from the chlorine gas drying from step g) and the dilute sulfuric acid from the nitration process of step b) are combined in a single sulfuric acid concentration plant and processed and part of the concentrated sulfuric acid is returned to the nitration process of step a) and part to the chlorine drying of step g) or to a further nitration plant producing a different aromatic nitro compound from the first nitration plant. This particular embodiment of the process allows, with a single sulfuric acid concentration plant, (i) chlorine to be dried, (ii) dinitrotoluene to be produced in the first nitration plant, and (iii) nitrobenzene to be produced in a second nitration plant, using the dilute sulfuric acid obtainable from the chlorine drying process, optionally before the concentration step k), as a catalyst in both nitrations. This process is advantageous if the amount of dilute sulfuric acid from the chlorine drying exceeds the sulfuric acid losses from one of the nitration processes. This in turn reduces the use of fresh sulfuric acid in the chlorine drying step g) by the recycled proportion.

Since different sulfuric acid concentrations may be required for catalysis of the nitration reaction and for chlorine drying, the sulfuric acid can be concentrated in several stages in order to provide the appropriate sulfuric acid concentration in the necessary amounts. Thus, for example, a sulfuric acid concentration of 86 to 91 percent by mass of sulfuric acid (based on the total mass of sulfuric acid plus water), as disclosed in EP-A 0903 336, may be needed for the nitration of toluene, whereas a higher concentration of 96.0 to 98.0 percent by mass of sulfuric acid (based on the total mass of sulfuric acid plus water) is advantageous for chlorine drying. In this case, the process according to the invention can be executed in such a way that the entire stream is concentrated in the sulfuric acid concentration plant to the concentration required for nitration and only the split stream required for chlorine drying is concentrated further in an additional distillation stage. In this way, the goal of a reduction in investment costs is also achieved.

Before sulfuric acid from a common acid concentration stage is returned to chlorine gas drying, purification steps can optionally be performed to remove any possibly disruptive components for the chlorine production process, such as organic nitro compounds, benzoic acids, nitrous gases or nitrosyl sulfuric acid. This can be done by means of adsorption or stripping methods.

The process according to the invention is particularly advantageous if toluene or benzene is used as the aromatic starting compound. Thus in the case of toluene, dinitrotoluene can be obtained after dinitration according to step a), which can then be reacted with hydrogen by any of the known processes in step c1) to form toluene diamine (TDA) and then reacted with phosgene by any of the known processes in step d) to form toluene diisocyanate (TDI).

In the case of dinitration to produce dinitrotoluene, in the second nitration stage, in which mononitrotoluene is nitrated further to form dinitrotoluene, a sulfuric acid concentration in the range from 86.0 to 96.0 percent by mass of sulfuric acid (based on the total mass of sulfuric acid plus water) is conventionally used before the addition of nitric acid. This sulfuric acid is then diluted with the water of reaction formed during nitration to a concentration of 80.0 to 85.9 percent by mass of sulfuric acid (based on the total mass of sulfuric acid plus water). The dilute sulfuric acid obtained from this second nitration stage with the concentration range from 80.0 to 85.9 percent by mass of sulfuric acid is then used as a feedstock sulfuric acid for the first nitration stage from toluene to mononitrotoluene, this sulfuric acid used being diluted in the course of this first nitration stage to a concentration of 70.0 to 79.9 percent by mass of sulfuric acid.

In the case of benzene, nitrobenzene is obtained by mononitration according to step a), the nitrobenzene is then reacted with hydrogen by any of the known processes to form aniline, which in turn is reacted with formaldehyde in the presence of an acid catalyst to form diamines and polyamines of the diphenylmethane series. The diamines and polyamines can then be reacted with phosgene by any of the known processes in step d) to form the corresponding di- and polyisocyanates of the diphenylmethane series.

In the nitration of benzene, before the addition of nitric acid, a sulfuric acid is used which has a concentration in the range from 69.5 to 72.5 percent by mass of sulfuric acid. On completion of nitration, the sulfuric acid obtained from this nitration has a concentration of 66.5 to 69.4 percent by mass of sulfuric acid.

Chlorine obtained by the process of the present invention can then be reacted with carbon monoxide by any of the known processes to form phosgene which can be used for the production of TDI or MDI from TDA or MDA, respectively. The hydrogen chloride produced during the phosgenation of TDA and MDA can then be reacted by any of the known processes to form chlorine.

Although the invention has been described in detail in the foregoing for the purpose of illustration, it is to be understood that such detail is solely for that purpose and that variations can be made therein by those skilled in the art without departing from the spirit and scope of the invention except as it may be limited by the claims.

What is claimed is:

1. A process for the coupled production of isocyanate(s) and chlorine comprising the following steps:
(1) nitrating an aromatic compound to form the corresponding mono- or dinitrated aromatic compound with nitric acid in the presence of sulfuric acid, the sulfuric acid having a concentration of from 65.0 to 98.0% by mass until the concentration of the sulfuric acid is reduced by from 0.5 to 25% by mass;
(2) transferring the reduced concentration sulfuric acid obtained from step (1) to a sulfuric acid concentration plant where it is concentrated;
(3) reacting the mono- or di-nitrated aromatic compound obtained from step (1) with hydrogen to form an amine or diamine;
(4) if the amine formed in (3) is aniline, further reacting the aniline with formaldehyde to form a diamine and/or polyamine of diphenylmethane;
(5) reacting the amine produced in step (3) or (4) with phosgene to form an isocyanate;
(6) transferring aqueous dissolved hydrogen chloride or gaseous hydrogen chloride formed in step (5) to a chlorine gas production unit;
(7) producing chlorine gas in the chlorine gas production unit;
(8) removing water of reaction or moisture from other sources from the chlorine gas from step (7) by treating the chlorine gas with a sulfuric acid having a concentration of 90.0 to 99.0 percent by mass of sulfuric acid until the sulfuric acid has a concentration of 65.0 to 90.0 percent by mass of sulfuric acid;
(9) reacting the chlorine gas from step (8) with carbon monoxide to produce phosgene;
(10) feeding the phosgene obtained from step (9) to the reaction conducted in step (5),
(11) transferring the sulfuric acid from step (8) either to (i) the sulfuric acid concentration plant of step (2) or (ii) to at least one nitration reaction being conducted in accordance with step (1) with subsequent transfer of further diluted sulfuric acid obtained from such nitration reaction to the sulfuric acid concentration plant of step (2);
(12) increasing concentration of the sulfuric acid streams from steps (2) and (8) combined in the sulfuric acid concentration plant by one or more vacuum distillation stages or by removing split streams after different distillation stages, as required for aromatic nitration in accordance with step (1) or for chlorine gas treatment in step (8); and
(13) returning the concentrated sulfuric acid from step (12) to step (8) and/or step (1).

2. The process of claim 1 in which step (7) is carried out using an NaCl electrolysis, an HCl electrolysis by a membrane or diaphragm method, an HCl electrolysis in an electrolytic cell with an oxygen depletion cathode, or catalytic HCl oxidation with oxygen process.

3. The process of claim 1 in which product of step (1) is nitrobenzene, dinitrobenzene, nitrochlorobenzene, nitrochlorotoluene, nitrotoluene or dinitrotoluene.

4. The process of claim 1 in which the sulfuric acid from step (8) is freed from residual chlorine and/or HCl down to a residual concentration of <1000 ppm Cl before combination in the sulfuric acid concentration plant according to step (12).

5. The process of claim 1 in which the sulfuric acid from step (8) is freed from residual chlorine and/or HCl down to a residual concentration of <10 ppm Cl before combination in the sulfuric acid concentration plant according to step (12).

6. The process of claim 1 in which the reduced concentration sulfuric acid from step (1) or the concentrated sulfuric acid from step (12) is freed from impurities using an inorganic nitrogen compound down to a residual concentration of <0.3 wt. % based on the total mass before step (13).

7. The process of claim 1 in which the reduced concentration sulfuric acid from step (1) or the concentrated sulfuric acid from step (12) is freed from impurities using a highly volatile organic compound down to a residual concentration of <50 ppm before step (13).

8. The process of claim 1 in which the reduced concentration sulfuric acid from step (1) or the concentrated sulfuric acid from step (12) is freed from impurities using a low-volatility organic compound down to a residual concentration of <500 ppm before step (13).

9. The process of claim 1 in which the reduced concentration sulfuric acid from step (1) or the concentrated sulfuric acid from step (12) is freed from impurities using a volatile sulfur compound down to a residual concentration of <30 ppm before step (13).

10. The process of claim 1 in which toluene is the aromatic compound used in step (1), sulfuric acid with a concentration of 86.0 to 96.0 percent by mass is used to produce mono-nitrotoluene, the concentration of the sulfuric acid is diluted to 80.0 to 85.9 percent by mass during nitration to form dinitrotoluene, the dinitrotoluene is reacted with hydrogen to form toluene diamine which is then reacted with phosgene to form toluene diisocyanate.

11. The process of claim 10 in which the sulfuric acid having a concentration of from 80.0 to 85.9 percent by mass is returned to the process for producing mononitrotoluene and the sulfuric acid concentration is further diluted to a concentration of 70.0 to 79.9 percent by mass of sulfuric acid before being fed to the sulfuric acid concentration plant in step (2).

12. The process of claim 1 in which benzene is the aromatic compound nitrated in step a) using sulfuric acid with a concentration of 69.5 to 72.5 percent by mass and which in the course of nitration to form nitrobenzene is diluted to a concentration of 66.5 to 69.4 percent by mass of sulfuric acid, the nitrobenzene is reacted with hydrogen to form aniline, which in turn is reacted with formaldehyde in the presence of an acid catalyst to form a diamine and/or polyamine of diphenylmethane, which in turn is reacted with phosgene to form a di- and/or polyisocyanate of diphenylmethane.
